(19) United States
(12) Patent Application Publication
(10) Pub. No.: US 2004/0137023 A1
(43) Pub. Date: Jul. 15, 2004

(54) 5-Cholesten-3beta, 25-diol-7-one, esters and ethers thereof, particularly for the treatment of dry skin and scalp

(75) Inventors: Maria Dalko, Gif S/Yvette (FR); Alexandre Cavezza, Tremblay-En-France (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/691,511

(22) Filed: Oct. 24, 2003

Related U.S. Application Data
(60) Provisional application No. 60/426,390, filed on Nov. 15, 2002.

(30) Foreign Application Priority Data
Oct. 24, 2002 (FR) 0213339

(51) Int. Cl. A61K 31/56; A61K 7/00

(52) U.S. Cl. 424/401; 514/177

(57) ABSTRACT

The present invention relates to a cosmetic process for treating dry skin or a dry scalp by the topical application to the skin or the scalp of a composition containing, in a physiologically acceptable medium, at least one of 5-cholesten-3β,25-diol-7-one and esters and ethers thereof.

### REFERENCE TO PRIOR APPLICATIONS

This application claims priority to U.S. provisional application 60/426,390 filed Nov. 15, 2002, and to French patent application 0213339 filed Oct. 24, 2002, both incorporated herein by reference.

### FIELD OF THE INVENTION

The present invention relates to a process for treating dry skin and/or a dry scalp, comprising the topical application to the skin and/or the scalp of a composition comprising, in a physiologically acceptable medium, at least one oxysterol selected from the group consisting of 5-cholesten-3β,25-diol-7-one and the esters and ethers thereof.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Many women of thirty five years old or more, and more particularly after the menopause, frequently complain of dryness of their skin, and of unaesthetic or uncomfortable manifestations resulting therefrom (desquamation, dull complexion, cutaneous atony). Now, as is now known, this dryness is caused by a decrease in the production of sebum with age.

Moreover, children whose sebaceous function is not yet active often show signs of dry skin.

Sebum is the natural product of the sebaceous gland, which, together with the sweat produced by the eccrine or apocrine glands, constitutes a natural moisturizer for the epidermis. It consists essentially of a more or less complex mixture of lipids. Conventionally, the sebaceous gland produces squalene, triglycerides, aliphatic waxes, cholesterol waxes and possibly free cholesterol (Stewart, M. E., Semin. Dermatol. 11, 100-105 (1992)). The action of bacterial lipases converts a variable portion of the triglycerides into free fatty acids.

Sebocytes are the competent cells of the sebaceous gland. The production of sebum is associated with the programme of terminal differentiation of these cells. During this differentiation, the metabolic activity of the sebocytes is essentially focused on the biosynthesis of lipids (lipogenesis) and more specifically on the neosynthesis of fatty acids and squalene.

A compound for stimulating the production of the lipids that form sebum, by the cells of the sebaceous gland (the sebocytes), would therefore be of definite advantage for the treatment of oligoseborrhoeic dry skin, i.e. skin with a sebum content of less than 100 μg/cm² on the forehead.

To this end, it has been proposed in patent U.S. Pat. No. 4,496,556 to use DHEA, a steroid secreted by the adrenal glands, or its esters, administered topically, to increase the production of sebum.

However, due to regulatory matters, it is not always possible to use compounds of this type in cosmetics. In addition, its efficacy is insufficient for oligoseborrhoeic skin. There is thus still a need for cosmetically acceptable compounds allowing the sebaceous function to be efficiently stimulated, for the purpose of treating oligoseborrhoeic dry skin.

Such compounds would also be useful in the treatment of a dry scalp, which is often associated with dull, lifeless hair.

### DETAILED DESCRIPTION OF THE PREFERRED EMBODIMENTS

The inventors have now discovered, surprisingly, that 5-cholesten-3β,25-diol-7-one and its esters and ethers satisfy this need.

Admittedly, it is known practice to use certain cholesterol derivatives for cosmetic purposes, in compositions applied topically to the skin. Thus, document FR-1 255 602 discloses compositions for preventing the dehydration of dry skin, caused in particular by age, comprising as adjuvant sterols such as 7-oxocholesterol. Document EP-0 608 600 also discloses compounds for replacing the skin lipids, which may also be used in the treatment of dry skin and a dry scalp, including 7-oxocholesterol.

Moreover, it has been suggested to use 25-hydroxycholesterol in cosmetic compositions intended especially for conditioning the skin and for combating wrinkles and skin roughness (JP-09 255 552 and JP-11 279 018).

However, to the inventor's knowledge, the cosmetic application of oxysterols such as 5-cholesten-3β,25-diol-7-one or esters or ethers of this compound (identified hereinbelow as “derivatives”) has as yet never been suggested.

One subject of the present invention is thus a cosmetic process for treating dry skin and/or a dry scalp, comprising the topical application to the skin and/or the scalp of a composition comprising, consisting essentially of, or consisting of, in a physiologically acceptable medium, at least one oxysterol selected from the group consisting of 5-cholesten-3β,25-diol-7-one and esters and ethers thereof.

5-Cholesten-3β,25-diol-7-one corresponds to formula (I) below:

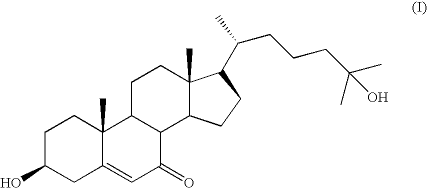

This compound is especially available from the company Steraloids under the reference C6550.

In a particular embodiment the esters and ethers are esters and ethers originating at one or both of the —OH moieties of formula (1), and preferably comprise 1-25 carbon atoms, more preferably 1-6 carbon atoms. When the ester derivative is depicted as —OCOR, R is preferably a branched, linear or cyclic alkyl group with optional unsaturation. Similarly, when the ether derivative is depicted as —OR, R is as defined for the above ester.

The composition according to the invention is particularly suitable for treating oligoseborrhoeic dry skin, i.e. skin with a sebum content of less than 100 μg/cm² on the forehead. This type of skin is frequently encountered in women around the time of the menopause, and as such the composition used according to the invention is preferably applied to women over forty.

Another subject of the present invention is the cosmetic use of at least one oxysterol selected from the group consisting of 5-cholesten-3β,25-diol-7-one and the esters and ethers thereof, as an agent for treating dry skin or a dry scalp.

A subject of the invention is also the use of at least one oxysterol selected from the group consisting of 5-cholesten-3β,25-diol-7-one and the esters and ethers thereof, for the preparation of a composition, especially a dermatological composition, for treating disorders associated with oligoseborrhoeic dry skin, in particular forms of dermatitis.

The amount of oxysterol that may be used according to the invention is not limited and preferably depends on the desired effect, and may thus vary within a wide range. In general, the oxysterol(s) will be present in an amount that is sufficient to significantly increase the production of sebum and advantageously to increase by at least 10% (including 12, 14, 16, 18, 20, 25, etc. % and more) the production of sebum by a culture of sebocytes, as described in Example 1 below.

To give an order of magnitude, these oxysterols may be used in an amount representing from, for example, 0.001% to 5% of the total weight of the composition, and preferably in an amount representing from 0.05% to 1% of the total weight of the composition.

The composition according to the invention is most preferably suitable for topical application to the skin and/or the scalp and thus contains a physiologically acceptable medium, i.e. a medium that is compatible with the skin, its integuments (eyelashes, nails and hair) and/or the mucous membranes. This medium may comprise water and optionally a water soluble alcohol, among other things.

The invention composition may be in any presentation form, including those normally used in cosmetics and dermatology, and it may especially be in the form of an optionally gelled oily solution, a dispersion of the lotion type, optionally a two-phase lotion, an emulsion obtained by dispersing a fatty phase in an aqueous phase (O/W emulsion) or conversely (W/o emulsion), or a triple emulsion (W/O/W or O/W/O emulsion) or a vesicular dispersion of ionic and/or nonionic type. These compositions are prepared according to the usual methods. A composition in the form of an oil-in-water emulsion is preferably used according to this invention.

This composition may be more or less fluid and may have the appearance of a white or coloured cream, an ointment, a milk, a lotion, a serum, a paste or a mousse. It may optionally be applied in the form of an aerosol. It may also be in solid form, in particular in the form of a stick. It may be used as a care product, and/or as a makeup product for the skin. It may also be used as a shampoo or conditioner.

In a known manner, the composition used according to the invention may also contain adjuvants such as those that are common in cosmetics, such as hydrophilic or lipophilic gelling agents, hydrophilic or lipophilic active agents, preserving agents, antioxidants, solvents, fragrances, fillers, screening agents, pigments, odour absorbers and dyestuffs. The amounts of these various adjuvants may be those conventionally used in the field under consideration, and, for example, from 0.01% to 20% relative to the total weight of the composition. Depending on their nature, these adjuvants may be introduced into the fatty phase, into the aqueous phase, or into lipid vesicles. In any case, these adjuvants, and also the proportions thereof, will be chosen so as not to harm the desired properties of the oxysterols used according to the invention.

When the composition used according to the invention is an emulsion, the proportion of the fatty phase may range from 5% to 80% by weight and preferably from 5% to 50% by weight relative to the total weight of the composition. The oils, emulsifiers and co-emulsifiers used in the composition in emulsion form may be selected from the group consisting of those conventionally used in the field under consideration. The emulsifier and co-emulsifier are preferably present in the composition in a proportion ranging from 0.3% to 30% by weight and preferably from 0.5% to 20% by weight relative to the total weight of the composition.

As oils which may be used in the invention, mention may be made of mineral oils (liquid petroleum jelly), oils of plant origin (avocado oil or soybean oil), oils of animal origin (lanolin), synthetic oils (perhydrosqualene), silicone oils (cyclomethicone) and fluoro oils (perfluoropolyethers). Fatty alcohols (cetyl alcohol), fatty acids and waxes (carnauba wax or ozokerite) may also be used as fatty substances.

As examples of emulsifiers and co-emulsifiers that may be used in the invention, mention may be made of, for example, fatty acid esters of polyethylene glycol such as PEG-100 stearate, and fatty acid esters of glycerol such as glyceryl stearate.

Hydrophilic gelling agents that may be mentioned in particular include carboxyvinyl polymers (carbomer), acrylic copolymers such as acrylate/alkyl-acrylate copolymers, polyacrylamides, polysaccharides, natural gums and clays, and lipophilic gelling agents that may be mentioned include modified clays, for instance bentones, metal salts of fatty acids, hydrophobic silica and polyethylenes.

As active agents, it will be advantageous to introduce into the composition used according to the invention at least one compound selected from the group consisting of: desquamating agents; moisturizers; calmatives; and agents for stimulating keratinocyte proliferation and/or differentiation.

Specifically, the stimulation of seborrhoea with the oxysterols and derivatives according to the invention may, in certain individuals, provide a proliferation terrain for the resident microflora of the follicular ostium (in particular Propionibacterium acnes), thus resulting in considerable hydrolysis of the sebum triglycerides to free fatty acids and the reduction of the unsaturations of polyunsaturated fatty acids (in particular linoleic acid). These two phenomena may contribute towards keratinization of the infundibulum and the formation of a micro-comedone. This may degenerate into a comedone, plugging and dilating the pore in an unattractive manner. At a more advanced stage, this plug may diverge towards an inflammatory acneic lesion.

The addition of desquamating agents or agents for regulating keratinocyte proliferation or differentiation to the composition according to the invention makes it possible to avoid the formation of these comedones. Similarly, antibacterial or bacteriostatic agents would, by modifying the proliferation of the resident microfloro, make it possible to obtain the same effect.

In addition, the moisturizers may complement the effect obtained using the oxysterol according to the invention, and the calmatives are useful for improving the comfort of oligoseborrhoeic dry skin.

Examples of such additional active agents are given below.

Desquamating Agents

The term “desquamating agent” means any compound capable of acting:

either directly on the desquamation by promoting exfoliation, such as β-hydroxy acids, in particular salicylic acid and its derivatives (including 5-n-octanoylsalicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol;

or on the enzymes involved in the desquamation or degradation of corneodesmosomes, glycosidases, stratum corneum chymotryptic enzyme (SCCE), or even other proteases (trypsin, chymotrypsin-like). Mention may be made of agents for chelating mineral salts: EDTA; N-acyl-N,N′,N′-ethylenediaminetriacetic acid; amino-sulphonic compounds and in particular (N-2-hydroxy-ethylpiperazine-N-2-ethane)sulphonic acid (HEPES); derivatives of 2-oxothiazolidine-4-carboxylic acid (procysteine); derivatives of α-amino acids of the type such as glycine (as described in EP-0 852 949, and sodium methylglycinediacetate sold by BASF under the trade name Trilon M); honey; sugar derivatives such as O-octanoyl-6-D-maltose and N-acetylglucosamine.

Moisturizer

The term “moisturizer” means:

either a compound that acts on the barrier function, in order to maintain the moisturization of the stratum corneum, or an occlusive compound. Mention may be made of ceramides, sphingoid-based compounds, lecithins, glycosphingolipids, phospholipids, cholesterol and its derivatives, phytosterols (stigmasterol, β-sitosterol or campesterol), essential fatty acids, 1,2-diacyl-glycerol, 4-chromanone, pentacyclic triterpenes such as ursolic acid, petroleum jelly and lanolin;

or a compound that directly increases the water content of the stratum corneum, such as threalose and its derivatives, hyaluronic acid and its derivatives, glycerol, pentanediol, sodium pidolate, serine, xylitol, sodium lactate, polyglyceryl acrylate, ectoin and its derivatives, chitosan, oligosaccharides and polysaccharides, cyclic carbonates, N-lauroyl-pyrrolidonecarboxylic acid and N-α-benzoyl-L-arginine;

or a compound that activates the sebaceous glands, such as DHEA and its derivatives; and vitamin D and its derivatives.

Agents for Stimulating Keratinocyte Proliferation and/or Differentiation

The agents for stimulating keratinocyte proliferation that may be used in the composition according to the invention include retinoids such as retinol and its esters, including retinyl palmitate; phloroglucinol; the walnut cake extracts sold by the company Gattefosse; and the Solanum tuberosum extracts sold by the company Sederma.

Retinoids are preferably used in this invention, in particular retinol and its esters.

The agents for stimulating keratinocyte differentiation comprise, for example, minerals such as calcium; the extract of lupin sold by the company Silab under the trade name Photopreventine®; sodium beta-sitosteryl sulphate sold by the company Seporga under the trade name Phytocohesine®; and the extract of corn sold by the company Solabia under the trade name Phytovityl®.

Calmatives

Among the raw materials that are effective as calmatives, mention may be made, in a non-limiting manner, of the following active agents: pentacyclic triterpenes, for instance β-glycyrrhetinic acid, its salts and/or its derivatives (glycyrrhetinic acid monoglucuronide, stearyl glycyrrhetinate, 3-stearoyloxyglycyrrhetic acid), ursolic acid and its salts, oleanolic acid and its salts, betulinic acid and its salts; extracts of Paeonia suffruticosa and/or lactiflora, of Rosmarinus officinalis, of epilobium, of Pygeum, of Boswellia serrata, of Centipeda cunninghami, of Helianthus annuus, of Cola nitida, of clove and of Bacopa moniera; salicylic acid salts and in particular zinc salicylate; extracts of algae, in particular of Laminaria saccharina; Canola oil, Tamanu oil, beauty-leaf oil, omega-3-unsaturated oils such as musk rose oil, blackcurrant oil, ecchium oil, fish oil; α-bisabolol and extracts of camomile; allantoin; the phosphoric diester of vitamins E and C; capryloyl-glycine; tocotrienols; piperonal; aloe vera; phytosterols; cortisone, hydrocortisone, indomethacin and beta-methasone.

Mention may also be made of substance P antagonists and especially strontium salts; spring waters and in particular the spring water of the Vichy basin and the spring water of La Roche Posay; bacterial extracts and in particular the extract of non-photosynthetic filamentous bacteria described in patent application EP-0 761 204, preferably prepared from bacteria belonging to the order Beggiatoales, more particularly to the genus Vitreoscilla. A strain of Vitreoscilla filiformis is preferably used according to the invention.

Mention may also be made of CGRP antagonists and in particular an extract of (preferably undifferentiated) cells of at least one plant from the Iridacea family, obtained by in vitro culturing. The Iridacea plant preferably belongs to the genus Iris. In particular, it is preferred to use an aqueous extract of Iris pallida, as described in patent application EP-0 765 668.

Finally, mention may be made of bradykinin antagonists and in particular an extract of at least one plant of the Rosaceae family, preferably cultivated in vivo. Preferably, a plant belonging to the genus Rosa, advantageously of the species Rosa gallica, more preferably an aqueous-glycol extract of Rosa gallica petals, as described in patent application EP-0 909 556, is used according to the invention.

Antibacterial Agents

The antibacterial agents that may be used in the present invention may be chosen especially from 2,4,4′-trichloro-2′-hydroxydiphenyl ether (or triclosan), 3,4,4′-trichlorocarbanilide, phenoxyethanol, phenoxypropanol, phenoxyisopropanol, hexamidine isethionate, metronidazole and its salts, miconazole and its salts, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, octopirox, octoxyglycerine, octanoylglycine, caprylyl glycol, 10-hydroxy-2-decanoic acid, dichlorophenyl imidazole dioxolane and its derivatives, described in patent WO 93/18743, farnesol and phytosphingosines, and mixtures thereof.

The preferred antibacterial agents are triclosan, phenoxyethanol, octoxyglycerine, octanoylglycine, 10-hydroxy-2-decanoic acid, caprylyl glycol, farnesol and azelaic acid.

The invention will now be illustrated with the non-limiting examples that follow. In these examples, the amounts are indicated as percentages by weight.

### EXAMPLES

### Example 1

### Demonstration of the Activity of 5-cholesten-3β,25-diol-7-one on Lipogenesis

5-Cholesten-3β,25-diol-7-one was tested, in comparison with 7-oxocholesterol and 25-hydroxy-cholesterol, on a model of immortalized human sebocytes in culture, derived from the SZ95 line described in Zouboulis, C. C., Seltmann, H., Neitzel, H. and Orfanos, C. E., Establishment and Characterization of an Immortalized Human Sebaceous Gland Cell Line, J. Invest. Dermatol., 113, 1011-1020 (1999).

The test consisted in measuring the amount of lipids produced by the sebocytes of the line (at confluence), in the presence or absence of active agents diluted in DMSO, such that the final amount of DMSO in the culture medium is 0.1%. After treatment for 2 days, the adherent cells are treated with Nile red (1 μg/ml). The lipid content is then quantified by measuring the fluorescence of the dye (excitation/emission pair: 485-540 nm for the neutral lipids).

The tests are performed in sixplicate and the experiment is repeated four times.

In parallel, proliferation tests (MUH) and cell viability tests (LDH) make it possible to check that the effects obtained are not associated with an appreciable change in these biological parameters.

The results are collated in the table below:

| CONCENTRATION OF TEST | LIPID VARIATION (relative |
|---|---|
| COMPOUND | to the control) |
| 5-Cholesten-3β,25-diol-7-one | +90% |
| (10⁻⁴ M) | |
| 5-Cholesten-3β,25-diol-7-one | +85% |
| (10⁻⁵ M) | |
| 7-Oxocholesterol (10⁻⁴ M) | +20% |
| 25-OH cholesterol (10⁻⁴ M) | 0% |

As is seen from this table, 5-cholesten-3β,25-diol-7-one induces a large increase in sebocytic lipogenesis, which is markedly greater than that induced with other cholesterol derivatives, even at a lower concentration than the latter derivatives.

In addition, in the same test, DHEA, tested at a concentration of 10⁻⁵ M, gave only a 13% increase in the lipid content of the sebum. The oxysterols according to the invention are therefore more efficient than DHEA.

### Example 2

### Cosmetic Composition (O/W Emulsion)

This composition is prepared in a manner that is conventional for those skilled in the art. The amounts given in these examples are indicated as percentages by weight.

| | |
|---|---|
| 5-Cholesten-3β,25-diol-7-one | 0.3% |
| Freeze-dried extract of rosemary | 0.2% |
| Glyceryl stearate | 2% |
| Polysorbate 60 | 1% |
| Stearic acid | 1.4% |
| Triethanolamine | 0.7% |
| Carbomer | 0.4% |
| Olive oil | 12% |
| Liquid fraction of shea butter | 12% |
| Octyldodecanol | 6% |
| Isononyl isononanoate | 10% |
| Antioxidant | 0.05% |
| Fragrance | 0.5% |
| Preserving agent | 0.3% |
| Water | qs 100% |

This composition, applied twice daily, reinvigorates the sebaceous function of dry skin.

The above written description of the invention provides a manner and process of making and using it such that any person skilled in this art is enabled to make and use the same, this enablement being provided in particular for the subject matter of the appended claims, which make up a part of the original description, and including a cosmetic process for treating dry skin or a dry scalp, comprising the topical application to the skin or the scalp of a composition containing, in a physiologically acceptable medium, at least one oxysterol selected from the group consisting of 5-cholesten-3β,25-diol-7-one and esters and ethers thereof.

Similarly enabled is a cosmetic use of at least one oxysterol selected from the group consisting of 5-cholesten-3β,25-diol-7-one and the esters and ethers thereof, as an agent for treating dry skin or a dry scalp, and the use of at least one oxysterol selected from the group consisting of 5-cholesten-3β,25-diol-7-one and the esters and ethers thereof, for treating one or more disorders associated with oligoseborrhoeic dry skin, such as forms of dermatitis, and for the preparation of a composition for treating disorders associated with oligoseborrhoeic dry skin.

As used above, the phrase “selected from the group consisting of” includes mixtures of the specified materials.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

What is claimed is:

1. A process for treating dry skin and/or dry scalp, comprising the topical application thereto of a composition comprising, in a physiologically acceptable medium, at least one oxysterol selected from the group consisting of 5-cholesten-3β,25-diol-7-one and esters and ethers thereof.

2. The process according to claim 1, wherein the oxysterol is 5-cholesten-3β,25-diol-7-one.

3. The process according to claim 1, wherein the oxysterol represents from 0.05% to 1% of the total weight of the composition.

4. The process according to claim 1, wherein application is to oligoseborrhoeic dry skin.

5. The process according to claim 1, wherein the said composition is applied to the dry skin and/or dry scalp of a women over forty years old.

6. A process for treating for treating one or more disorders associated with oligoseborrhoeic dry skin comprising applying to oligoseborrhoeic dry skin a composition comprising, in a physiologically acceptable medium, at least one oxysterol selected from the group consisting of 5-cholesten-3β,25-diol-7-one and esters and ethers thereof

7. The process according to claim 6, wherein the said disorders are forms of dermatitis.

8. A composition comprising, in a physiologically acceptable medium, at least one oxysterol selected from the group consisting of 5-cholesten-3β,25-diol-7-one and the esters and ethers thereof, and at least one compound selected from the group consisting of a desquamating agent, a moisturizer, a calmative, an agent for promoting keratinocyte proliferation and/or differentiation, and an antibacterial agent.

9. The composition according to claim 8, comprising a desquamating agent.

10. The composition according to claim 9, wherein the said desquamating agent is selected from the group consisting of: salicylic acid; α-hydroxy acids; urea; gentisic acid; oligofucoses; cinnamic acid; extract of Saphora japonica; resveratrol; EDTA; N-acyl-N,N′,N′-ethylenediaminetriacetic acid; aminosulphonic compounds; derivatives of 2-oxothiazolidine-4-carboxylic acid (procysteine); honey; O-octanoyl-6-D-maltose and N-acetylglucosamine.

11. The composition according to claim 8, comprising a moisturizer selected from the group consisting of: ceramides, sphingoid-based compounds, lecithins, glycosphingolipids, phospholipids, cholesterol and its derivatives, phytosterols, essential fatty acids, 1,2-diacylglycerol, 4-chromanone, pentacyclic triterpenes; threalose and its derivatives, hyaluronic acid and its derivatives, glycerol, pentanediol, sodium pidolate, serine, xylitol, sodium lactate, polyglyceryl acrylate, ectoin and its derivatives, chitosan, oligosaccharides and polysaccharides, cyclic carbonates, N-lauroyl-pyrrolidonecarboxylic acid and N-α-benzoyl-L-arginine; DHEA and its derivatives; and vitamin D and its derivatives.

12. The composition according to claim 8, comprising a calmative selected from the group consisting of: pentacyclic triterpenes; extracts of Paeonia suffruticosa and/or lactiflora, of Rosmarinus officinalis, of epilobium, of Pygeum, of Boswellia serrata, of Centipeda cunninghami, of Helianthus annuus, of Cola nitida, of clove and of Bacopa moniera; salicylic acid salts; extracts of algae; Canola oil, Tamanu oil, beauty-leaf oil, omega-3-unsaturated oils such as musk rose oil, blackcurrant oil, ecchium oil, fish oil; α-bisabolol and extracts of camomile; allantoin; the phosphoric diester of vitamins E and C; capryloylglycine; tocotrienols; piperonal; aloe vera; phytosterols; cortisone, hydrocortisone, indomethacin and beta-methasone; a strain of Vitreoscilla filiformis, an aqueous extract of Iris pallida; and an aqueous-glycol extract of Rosa gallica petals.

13. The composition according to claim 8, comprising an agent for promoting keratinocyte proliferation that is a retinoid.

14. The composition according to claim 8, comprising an antibacterial agent selected from the group consisting of: triclosan, phenoxyethanol, octoxyglycerol, octanoylglycine, 10-hydroxy-2-decanoic acid, caprylyl glycol, farnesol and azelaic acid.

15. The Composition according to claim 8, wherein said composition is in the form of an oil-in-water emulsion.

16. The Composition according to claim 10, wherein said composition is in the form of an oil-in-water emulsion.

17. The Composition according to claim 11, wherein said composition is in the form of an oil-in-water emulsion.

18. The Composition according to claim 12, wherein said composition is in the form of an oil-in-water emulsion.

19. The Composition according to claim 13, wherein said composition is in the form of an oil-in-water emulsion.

20. The Composition according to claim 14, wherein said composition is in the form of an oil-in-water emulsion.
